(19) United States
(12) Patent Application Publication
(10) Pub. No.: US 2025/0345244 A1
(43) Pub. Date: Nov. 13, 2025

(54) INTELLIGENT MEDICATION DISPENSING BOX

(71) Applicant: JINGHAN ZHENG, SHENZHEN (CN)

(72) Inventor: JINGHAN ZHENG, SHENZHEN (CN)

(21) Appl. No.: 19/275,505

(22) Filed: Jul. 21, 2025

(30) Foreign Application Priority Data
Jun. 26, 2025 (CN) 2025108698310

(51) Int. Cl. A61J 7/00; A61J 1/03; A61J 7/02; A61J 7/04; G16H 20/13

(52) U.S. Cl. CPC A61J 7/0084; A61J 1/03; A61J 7/02; A61J 7/0454; G16H 20/13; A61J 2200/70

(57) ABSTRACT

The present disclosure belongs to the technical field of intelligent medical equipment, and in particular to an intelligent medication dispensing box, comprising an outer liner, wherein an inner liner is movably disposed inside the outer liner, and a plurality of medication chambers are movably disposed inside the inner liner; a bottom of the outer liner is provided with an inner liner base. The present disclosure replaces the high-cost servo system with the positioning magnets, positioning of the first Hall switch and the mechanism of the gear and the medication ejection rack to achieve accurate medication dispensing and rotation positioning, significantly reducing manufacturing costs and improving mechanical reliability. Sound and light alarm and button-free design are adopted to avoid maloperation, greatly reducing the threshold for use by elderly users. The split-type independent medication chambers support flexible replacement of different specifications and adapt to various medication needs.

### TECHNICAL FIELD

The present disclosure relates to the technical field of intelligent medical equipment, and in particular to an intelligent medication dispensing box.

### BACKGROUND

A medication box is a container for packaging pharmaceuticals. In daily use, a reminder medication box is also referred to as timed medication box or electronic medication box. It is a household electronic reminder and medication dispensing device designed for drug storage and equipped with alert functions to ensure timely medication intake.

With the intensification of population aging and the increasing prevalence of chronic diseases, accurate and timely medication intake has become a daily necessity for many individuals. Conventional medication boxes offer limited functionality, merely providing storage capacity without precision dosage sorting, scheduled reminders, or error-proof mechanisms. These deficiencies frequently lead to missed, incorrect, or duplicated doses, ultimately compromising therapeutic efficacy. While some intelligent medication boxes incorporate reminder functions, most employ structurally complex or cost-prohibitive solutions that hinder widespread adoption, especially in household or personal daily use.

Therefore, an intelligent medication dispensing box is provided to solve the above problems.

### SUMMARY OF THE APPLICATION

### I. Technical Issues Solved

In view of the deficiencies in the prior art, the present disclosure provides an intelligent medication dispensing box, which solves the problems in the above background technology that accurate medicine dispensing, timed reminders and maloperation prevention are not possible, and the degree of intelligence is low.

### II. Technical Solutions

In order to achieve the above objectives, the present disclosure specifically adopts the following technical solutions:
An intelligent medication dispensing box, comprising an outer liner, wherein an inner liner is movably disposed inside the outer liner, and a plurality of medication chambers are movably disposed inside the inner liner;
A bottom of the outer liner is provided with an inner liner base, a top of the inner liner base is provided with an inner liner middle cylinder of which a top end extends into an interior of the inner liner, an interior of the inner liner base is provided with a medication distributing servo, and an output end of the medication distributing servo is provided with a connecting shaft of which a top end penetrates through the inner liner middle cylinder and is fixed to the interior of the inner liner;
A medication outlet is disposed on one side of the inner liner middle cylinder, and a medication receiving box of which one end is located below the medication outlet is movably disposed at a bottom of the inner liner base;
An ejection mechanism base is disposed on one side of the outer liner, an ejection servo is disposed inside the ejection mechanism base, and an output end of the ejection servo is in transmission connection with a medication ejection rack.

In some embodiments, a width of an inner wall of the medication chamber increases from top to bottom, a bottom of the medication chamber is provided with a medication outlet hole with two ends penetrating to two sides of the medication chamber, and a bottom of an inner wall of the medication chamber is provided with a groove located at a bottom of the medication outlet hole.

In some embodiments, a bottom of two sides and a bottom of the medication chamber are respectively provided with a fool-proof limiting groove and a retrieval groove, the plurality of the medication chambers are distributed at equal intervals in a circular shape inside the inner liner, and a plurality of ventilation holes located between the two fool-proof limiting grooves are disposed on two sides of the medication chamber.

In some embodiments, an output end of the ejection servo is provided with a gear meshing with the medication ejection rack, and one end of the medication ejection rack is circular and matches the medication outlet hole.

In some embodiments, a plurality of positioning magnets which are the same as the count of the medication chambers and one-to-one corresponding to the medication chambers are disposed at a bottom of the inner liner, the bottom of the inner liner is provided with a zero position magnet located at a bottom of one of the medication chambers, a control base is disposed at a bottom of the ejection mechanism base, and a first Hall sensor and a second Hall sensor are disposed at a top of the control base and correspond to the positioning magnet and the zero position magnet, respectively.

In some embodiments, a transformer module is disposed at a bottom of an inner wall of the control base, and a loudspeaker and a main control board are respectively disposed on two sides of the inner wall of the control base.

In some embodiments, a photoelectric counter is disposed inside the medication outlet, the medication outlet is opposite to a position of the medication ejection rack, a control cover is fixedly disposed at a top of the ejection mechanism base, and a display screen is disposed inside the control cover.

In some embodiments, a bearing is sleeved between the connecting shaft and the inner liner middle cylinder, and an upper cover is movably disposed at a top of the outer liner.

In some embodiments, a bottom of the outer liner is fixedly connected to a top of the inner lining base through an outer liner fixing ring, a bottom of the inner lining base is fixedly provided with a servo fixing ring, and a top of the servo fixing ring is fixedly connected to a bottom of the medication dispensing servo.

In some embodiments, a base is fixedly disposed at a bottom of the servo fixing ring, a medication box limiting groove is disposed inside the base, the medication receiving box is movably disposed inside the medication box limiting groove, and one end of the medication receiving box extends to an interior of the base and is provided with a handle.

### III. Beneficial Effects

Compared with the prior art, the present disclosure provides an intelligent medication dispensing box, which has the following beneficial effects:

The present disclosure replaces the high-cost servo system with the positioning magnets, positioning of the first Hall switch and the mechanism of the gear and the medication ejection rack to achieve accurate medication dispensing and rotation positioning, significantly reducing manufacturing costs and improving mechanical reliability. Sound and light alarm and button-free design are adopted to avoid maloperation, greatly reducing the threshold for use by elderly users. The split-type independent medication chambers support flexible replacement of different specifications and adapt to various medication needs. It is combined with the fool-proof limiting groove to ensure accurate mounting, and the ventilation holes maintain a moisture-proof environment for medications, which comprehensively solves the problems of missed and wrong medications and moisture in conventional medication boxes. The intelligent medication dispensing box is simple and compact in structure, meets economic benefits, and has broad application prospects.

### BRIEF DESCRIPTION OF THE DRAWINGS

FIG. 1 is a schematic structural diagram of an outer liner according to the present disclosure;

FIG. 2 is a side view of an outer liner according to the present disclosure;

FIG. 3 is a schematic structural diagram of a medication receiving box according to the present disclosure;

FIG. 4 is a schematic structural diagram of an inner liner according to the present disclosure;

FIG. 5 is a schematic structural diagram of a medication chamber according to the present disclosure;

FIG. 6 is a schematic structural diagram of a positioning magnet and a zero position magnet according to the present disclosure;

FIG. 7 is a schematic structural diagram of a servo fixing ring according to the present disclosure;

FIG. 8 is a cross-sectional view of a medication chamber according to the present disclosure.

Reference signs in the figures: 1. outer liner; 11. upper cover; 12. outer liner fixing ring; 2. inner liner; 21. positioning magnet; 22. zero position magnet; 3. medication chamber; 31. medication outlet; 32. groove; 33. fool-proof limiting groove; 34. retrieval groove; 35. ventilation hole; 4. inner liner base; 41. inner liner middle cylinder; 42. medication dispensing servo; 43. connecting shaft; 44. medication outlet; 45. photoelectric counter; 46. bearing; 5. medication receiving box; 51. handle; 6. ejection mechanism base; 61. ejection servo; 62. medication ejection rack; 63. gear; 64. control cover; 65. display screen; 7. control base; 71. first Hall sensor; 72. second Hall sensor; 73. transformer module; 74. loudspeaker; 75. main control board; 8. servo fixing ring; 9. base; 91. medication box limiting groove.

### DETAILED DESCRIPTION

The following will clearly and completely describe the technical solutions in the embodiments of the present disclosure with reference to the drawings in the embodiments of the present disclosure. Obviously, the described embodiments are only part of the embodiments of the present disclosure, not all of the embodiments. Based on the embodiments of the present disclosure, all other embodiments obtained by those having ordinary skills in the art without creative work are within the scope of protection of the present disclosure.

### Embodiments

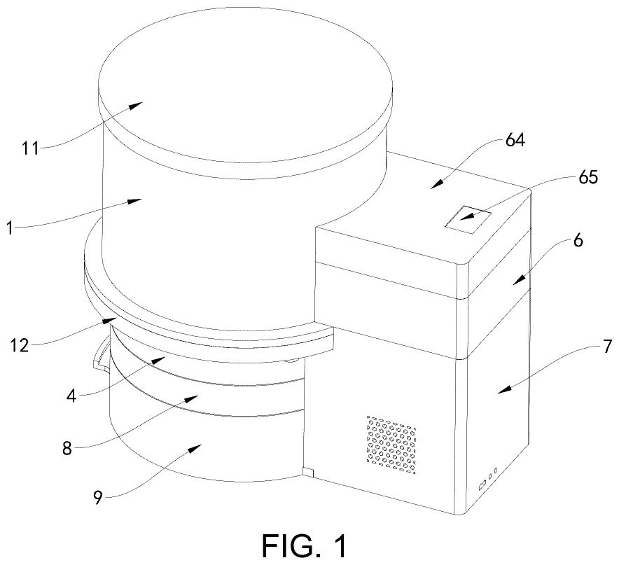

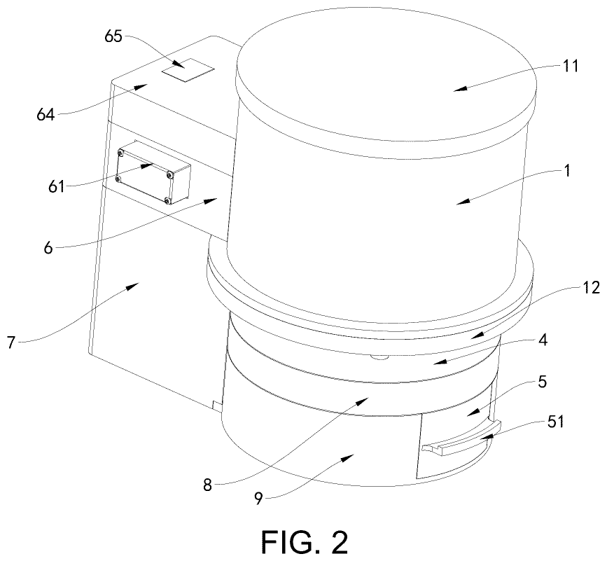

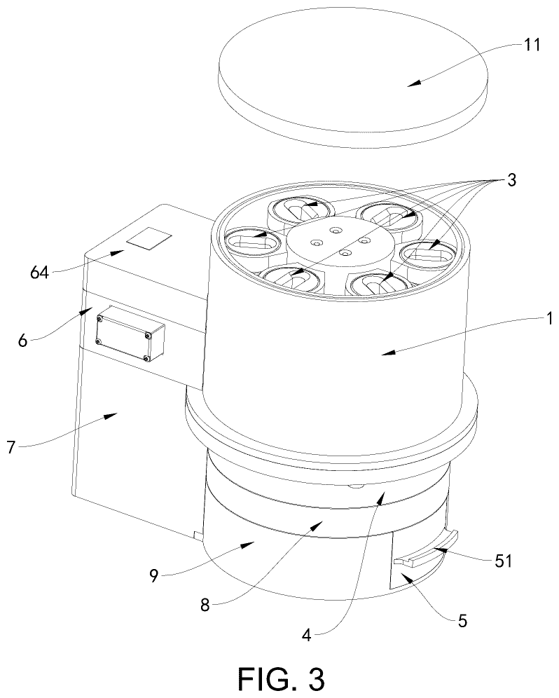

As shown in FIGS. 1-8, an intelligent medication dispensing box provided in one embodiment of the present disclosure includes an outer liner 1. An inner liner 2 is movably disposed inside the outer lining 1, and a plurality of medication chambers 3 are movably disposed inside the inner liner 2.

A bottom of the outer liner 1 is provided with an inner liner base 4. A top of the inner liner base 4 is provided with an inner liner middle cylinder 41 of which a top end extends into an interior of the inner liner 2. An interior of the inner liner base 4 is provided with a medication distributing servo 42. An output end of the medication distributing servo 42 is provided with a connecting shaft 43 of which a top end penetrates through the inner liner middle cylinder 41 and is fixed to the interior of the inner liner 2.

A medication outlet 44 is disposed on one side of the inner liner middle cylinder 41, and a medication receiving box 5 of which one end is located below the medication outlet 44 is movably disposed at a bottom of the inner liner base 4.

An ejection mechanism base 6 is disposed on one side of the outer lining 1. An ejection servo 61 is disposed inside the ejection mechanism base 6, and an output end of the ejection servo 61 is in transmission connection with a medication ejection rack 62.

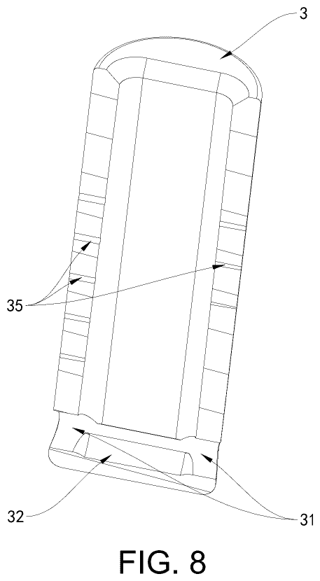

As shown in FIG. 8, in some embodiments, a width of an inner wall of the medication chamber 3 increases from top to bottom. A bottom of the medication chamber 3 is provided with a medication outlet hole 31 with two ends penetrating to two sides of the medication chamber 3, and a bottom of an inner wall of the medication chamber 3 is provided with a groove 32 located at a bottom of the medication outlet hole 31.

The increasing width of the inner wall of the medication chamber 3 helps the medication to fall naturally and be positioned near the medication outlet hole 31 without getting stuck during the process. Combined with the design of the groove 32, the medication will not fall directly from the medication outlet hole 31, ensuring that the pills are not easily stuck or offset when being ejected out, thereby accurately controlling that only one pill is ejected out each time, reducing the risk of missing or overdosing. Meanwhile, the structure simplifies the internal design of the medication chamber 3 and supports low cost and high reliability.

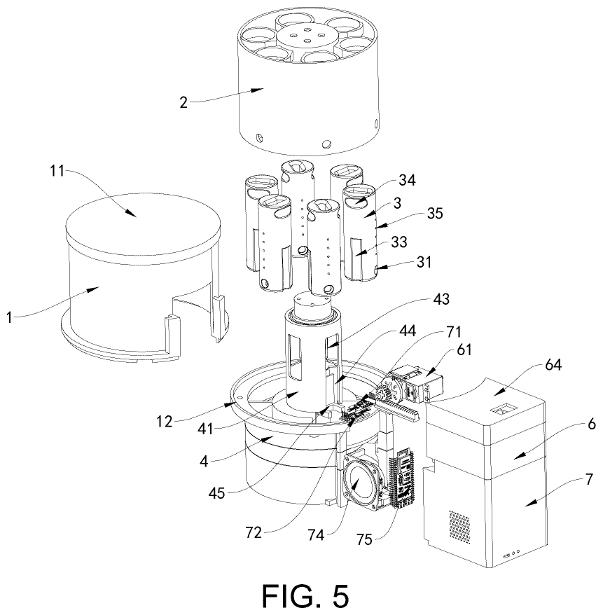

As shown in FIG. 5, in some embodiments, a bottom of two sides and a bottom of the medication chamber 3 are respectively provided with a fool-proof limiting groove 33 and a retrieval groove (34). The plurality of the medication chambers 3 are distributed at equal intervals in a circular shape inside the inner liner 2. A plurality of ventilation holes 35 located between the two fool-proof limiting grooves (33) are disposed on two sides of the medication chamber 3.

The fool-proof limiting groove 33 prevents the medication chamber 3 from being mounted in the wrong position, ensuring the correct assembly of the split structure. The retrieval groove 34 facilitates a user to easily operate the medication chamber 3 to replace it, improving the simple interactivity. The circular equidistant distribution optimizes the space utilization, increases the storage capacity, and improves the compactness of the structure. The ventilation holes 35 promote the internal air circulation, maintain the dry environment of the medication box, prevent the medication from getting damp, and enhance the compatibility and safety performance.

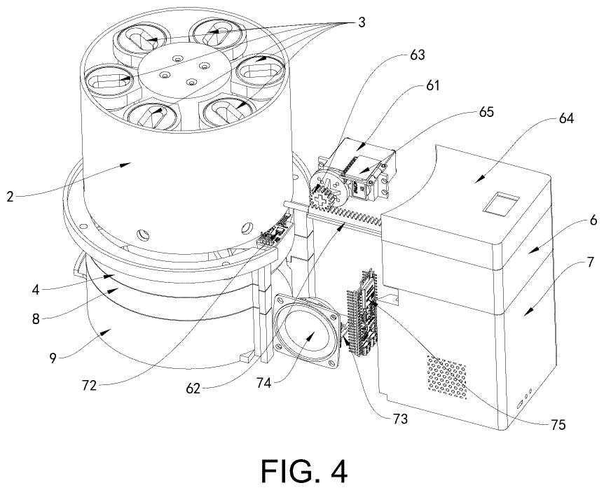

As shown in FIG. 4, in some embodiments, an output end of the ejection servo 61 is provided with a gear 63 meshing with the medication ejection rack 62. One end of the medication ejection rack 62 is circular and matches the medication outlet hole 31.

The meshing mechanism of the gear 63 and the rack realizes a smooth and reliable ejection action, replacing the high-cost solution of the servo motor and reducing the manufacturing costs. The circular end of the medication ejection rack 62 matches the shape of the medication outlet hole 31 to ensure accurate medication ejection, prevent pill damage or ejection failure, improve the accuracy of medication dispensing, and reduce the risk of maloperation.

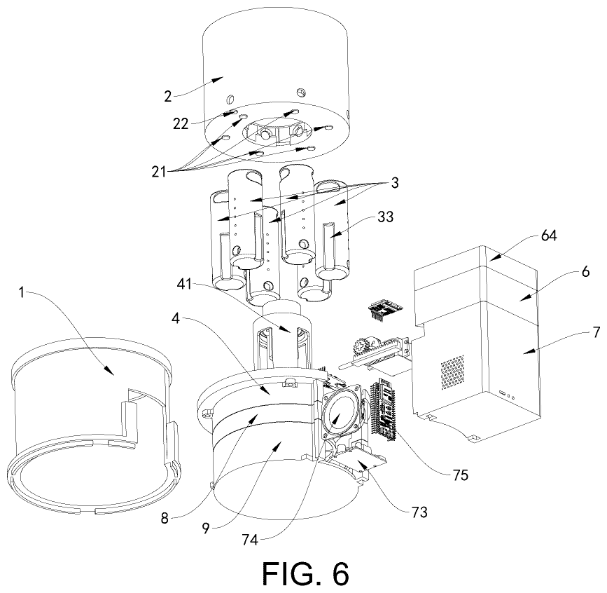

As shown in FIG. 6, in some embodiments, a plurality of positioning magnets 21 which are the same as the count of the medication chambers 3 and one-to-one corresponding to the medication chambers 3 are disposed at a bottom of the inner liner. The bottom of the inner liner 2 is provided with a zero position magnet 22 located at a bottom of one of the medication chambers 3. A control base 7 is disposed at a bottom of the ejection mechanism base 6. A first Hall sensor 71 and a second Hall sensor 72 are disposed at a top of the control base 7 and correspond to the positioning magnet 21 and the zero position magnet 22, respectively.

The positioning magnet 21 and the first Hall sensor 71 cooperate to realize a magnetic positioning rotation system, accurately detecting the position of the medication chamber 3, thereby avoiding the wear problem of conventional mechanical limiting, and improving the reliability and service life. The zero position magnet 22 and the second Hall sensor 72 cooperate to mark the reference position of the medication chamber 3, and the power-on calibration prevents misalignment, ensuring that the medication dispensing process is accurate, thereby reducing the risk of wrong medications, and strengthening safety guarantee.

As shown in FIG. 6, in some embodiments, a transformer module 73 is disposed at a bottom of an inner wall of the control base 7. A loudspeaker 74 and a main control board 75 are respectively disposed on two sides of the inner wall of the control base 7.

The transformer module 73, the photoelectric counter 45, the loudspeaker 74, the first Hall sensor 71, the second Hall sensor 72, the display screen 65, the medication dispensing servo 42, the photoelectric counter 45 and the ejection servo 61 are electrically connected to the main control board 75. The transformer module 73 supports direct power supply via Type-C, provides stable power input, and simplifies user operation. The loudspeaker 74 issues a sound reminder to effectively avoid the problem of missed doses. The main control board 75 integrates control functions to facilitate timed reminders, photoelectric counting and remote smart medical equipment PP communication, thereby improving interactive friendliness and system intelligence, and meeting the requirements of simple interaction and remote reminders in the design goals.

As shown in FIG. 5, in some embodiments, the photoelectric counter 45 is disposed inside the medication outlet 44. The medication outlet 44 is opposite to a position of the medication ejection rack 62. A control cover 64 is fixedly disposed at a top of the ejection mechanism base 6. A display screen 65 is disposed inside the control cover 64.

Each time the medication is dispensed, it will pass through the photoelectric counter 45 at the medication outlet 44. The photoelectric counter 45 senses the real-time statistics and secondary detection of the amount of medication dispensed, and compares it with a set amount. If there is an abnormal excess or leakage, an alarm is triggered to prevent wrong or excessive medication, thereby enhancing safety guarantee. The position of the medication outlet 44 opposite to the medication ejection rack 62 ensures that the medication accurately slides into the medication receiving box 5. The display screen 65 provides local status display and supports simple interaction, which is convenient for the user to view information without complicated operations, thereby improving user friendliness.

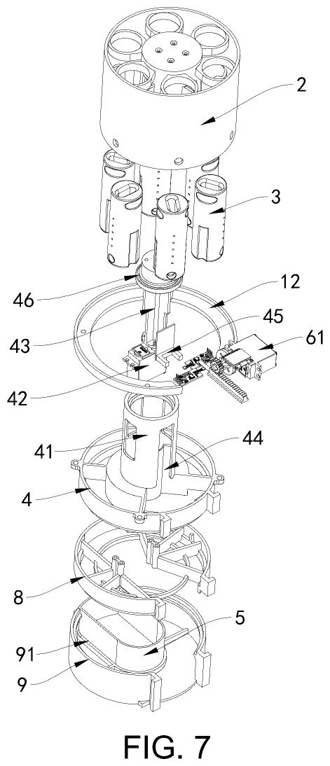

As shown in FIG. 7, in some embodiments, a bearing 46 is sleeved between the connecting shaft 43 and the inner liner middle cylinder 41. An upper cover 11 is movably disposed at a top of the outer liner 1.

The bearing 46 reduces the rotational friction of the connecting shaft 43, thereby ensuring the smooth rotation of the inner liner 2, and improving the mechanical reliability and durability. The upper cover 11 is movably arranged to protect the internal components, isolate external dust and moisture, maintain an internal closed moisture-proof environment, prevent the medication from getting damp, support the dryer upgrade, optimize the storage conditions of the medication, and facilitate the opening and replacement of the medication chamber 3 and the medication, respectively.

As shown in FIG. 5, in some embodiments, a bottom of the outer lining 1 is fixedly connected to a top of the inner lining base 4 through an outer lining fixing ring 12. A bottom of the inner lining base 4 is fixedly provided with a servo fixing ring 8. A top of the servo fixing ring 8 is fixedly connected to a bottom of the medication dispensing servo 42.

The outer liner fixing ring 12 and the servo fixing ring 8 provide stable connection to ensure the firmness of the overall structure and reduce the risk of maloperation caused by shaking. The servo fixing ring 8 accurately fixes the medication dispensing servo 42 to ensure the reliable operation of rotation positioning and ejection mechanism, thereby improving the mechanical stability of the system, extending the service life, and meeting the design goal of high reliability.

As shown in FIG. 7, in some embodiments, a base 9 is fixedly disposed at a bottom of the servo fixing ring 8. A medication box limiting groove 91 is disposed inside the base 9. The medication receiving box 5 is movably disposed inside the medication box limiting groove 91, and one end of the medication receiving box 5 extends to an interior of the base 9 and is provided with a handle 51.

The medication box limiting groove 91 guides the medication receiving box 5 to be accurately positioned to prevent the medication from spilling during the retrieval process. The handle 51 is configured to facilitate the user to easily extract the medication, simplifying the operation process. It is especially suitable for the elderly or children to avoid maloperation. The drawer-type medication retrieval structure ensures the safe retrieval of the medication and solves the problem of easy spillage of conventional medication boxes.

In summary, the high-cost servo system is replaced with the positioning magnets 21, positioning of the first Hall switch and the mechanism of the gear 63 and the medication ejection rack 62 to achieve accurate medication dispensing and rotation positioning, significantly reducing manufacturing costs and improving mechanical reliability. Sound and light alarm and button-free design are adopted to avoid maloperation, greatly reducing the threshold for use by elderly users. The split-type independent medication chambers 3 support flexible replacement of different specifications and adapt to various medication needs. It is combined with the fool-proof limiting groove 33 to ensure accurate mounting, and the ventilation holes 35 maintain a moisture-proof environment for medications, which comprehensively solves the problems of missed and wrong medications and moisture in conventional medication boxes. The intelligent medication dispensing box is simple and compact in structure, meets economic benefits, and has broad application prospects.

Finally, it should be noted that the above description is only a preferred embodiment of the present disclosure and is not intended to limit the present disclosure. Although the present disclosure has been described in detail with reference to the embodiments, those skilled in the art can still modify the technical solutions described in the embodiments or replace some of the technical features therein by equivalents. Any modification, equivalent replacement, improvement, etc. made within the spirit and principle of the present disclosure shall be included in the protection scope of the present disclosure.

1. An intelligent medication dispensing box, comprising an outer liner (1), wherein an inner liner (2) is movably disposed inside the outer liner (1), and a plurality of medication chambers (3) are movably disposed inside the inner liner (2);
a bottom of the outer liner (1) is provided with an inner liner base (4), a top of the inner liner base (4) is provided with an inner liner middle cylinder (41) of which a top end extends into an interior of the inner liner (2), an interior of the inner liner base (4) is provided with a medication distributing servo (42), and an output end of the medication distributing servo (42) is provided with a connecting shaft (43) of which a top end penetrates through the inner liner middle cylinder (41) and is fixed to the interior of the inner liner (2);
a medication outlet (44) is disposed on one side of the inner liner middle cylinder (41), and a medication receiving box (5) of which one end is located below the medication outlet (44) is movably disposed at a bottom of the inner liner base (4);
an ejection mechanism base (6) is disposed on one side of the outer liner (1), an ejection servo (61) is disposed inside the ejection mechanism base (6), and an output end of the ejection servo (61) is in transmission connection with a medication ejection rack (62).

2. The intelligent medication dispensing box according to claim 1, wherein a width of an inner wall of the medication chamber (3) increases from top to bottom, a bottom of the medication chamber (3) is provided with a medication outlet hole (31) with two ends penetrating to two sides of the medication chamber (3), and a bottom of an inner wall of the medication chamber (3) is provided with a groove (32) located at a bottom of the medication outlet hole (31).

3. The intelligent medication dispensing box according to claim 1, wherein a bottom of two sides and a bottom of the medication chamber (3) are respectively provided with a fool-proof limiting groove (33) and a retrieval groove (34), the plurality of the medication chambers (3) are distributed at equal intervals in a circular shape inside the inner liner (2), and a plurality of ventilation holes (35) located between the two fool-proof limiting grooves (33) are disposed on two sides of the medication chamber (3).

4. The intelligent medication dispensing box according to claim 1, wherein an output end of the ejection servo (61) is provided with a gear (63) meshing with the medication ejection rack (62), and one end of the medication ejection rack (62) is circular and matches the medication outlet hole (31).

5. The intelligent medication dispensing box according to claim 1, wherein a plurality of positioning magnets (21) which are the same as the count of the medication chambers (3) and one-to-one corresponding to the medication chambers (3) are disposed at a bottom of the inner liner (2), the bottom of the inner liner (2) is provided with a zero position magnet (22) located at a bottom of one of the medication chambers (3), a control base (7) is disposed at a bottom of the ejection mechanism base (6), and a first Hall sensor (71) and a second Hall sensor (72) are disposed at a top of the control base (7) and correspond to the positioning magnet (21) and the zero position magnet (22), respectively.

6. The intelligent medication dispensing box according to claim 5, wherein a transformer module (73) is disposed at a bottom of an inner wall of the control base (7), and a loudspeaker (74) and a main control board (75) are respectively disposed on two sides of the inner wall of the control base (7).

7. The intelligent medication dispensing box according to claim 1, wherein a photoelectric counter (45) is disposed inside the medication outlet (44), the medication outlet (44) is opposite to a position of the medication ejection rack (62), a control cover (64) is fixedly disposed at a top of the ejection mechanism base (6), and a display screen (65) is disposed inside the control cover (64).

8. The intelligent medication dispensing box according to claim 1, wherein a bearing (46) is sleeved between the connecting shaft (43) and the inner liner middle cylinder (41), and an upper cover (11) is movably disposed at a top of the outer liner (1).

9. The intelligent medication dispensing box according to claim 1, wherein a bottom of the outer liner (1) is fixedly connected to a top of the inner lining base (4) through an outer liner fixing ring (12), a bottom of the inner lining base (4) is fixedly provided with a servo fixing ring (8), and a top of the servo fixing ring (8) is fixedly connected to a bottom of the medication dispensing servo (42).

10. The intelligent medication dispensing box according to claim 9, wherein a base (9) is fixedly disposed at a bottom of the servo fixing ring (8), a medication box limiting groove (91) is disposed inside the base (9), the medication receiving box (5) is movably disposed inside the medication box limiting groove (91), and one end of the medication receiving box (5) extends to an interior of the base (9) and is provided with a handle (51).
